(19) United States
(12) Patent Application Publication
(10) Pub. No.: US 2025/0002957 A1
(43) Pub. Date: Jan. 2, 2025

(54) SYSTEM AND METHOD TO CREATE A WATER-SOLUBLE MIXTURE OF OLIGOSACCHARIDES FOR FACILE CONVERSION TO FERMENTABLE SUGARS

(71) Applicant: BLUE BIOFUELS, INC., Palm Beach Gardens, FL (US)

(72) Inventors: Benjamin SLAGER, Delray Beach, FL (US); Eric R. LIBRA, North Palm Beach, FL (US); Travis Wayne BAUGHMAN, North Palm Beach, FL (US)

(73) Assignee: BLUE BIOFUELS, INC., Palm Beach Gardens, FL (US)

(21) Appl. No.: 18/274,138

(22) PCT Filed: Mar. 28, 2023
(86) PCT No.: PCT/US2023/016586

§ 371 Date: Jul. 25, 2023

Related U.S. Application Data
(60) Provisional application No. 63/304,027, filed on Jan. 28, 2022.

(51) Int. Cl. C12P 19/14; C12M 1/40; C12N 9/26; C12N 9/42; C12P 19/02; C12P 19/12

(52) U.S. Cl. CPC C12P 19/14; C12M 21/18; C12N 9/2411; C12N 9/2437; C12P 19/02; C12P 19/12; C12P 2201/00; C12Y 302/01004

(57) ABSTRACT

A system and method for generating a water-soluble mixture of oligosaccharides for facile conversion to fermentable sugars is provided. The method describes crushing, grinding, or both, a mixture of a cellulose feedstock and a solid acid catalyst, under pressure to induce a solid-solid interaction between the cellulosic feedstock and the solid acid catalyst to induce a chemical reaction to produce a grinded mixture, wherein the crusher assembly comprises rollers,

introducing water to separate the grinded mixture into solids and a solution, wherein the solution comprises the oligosaccharides and enzymatically converting the solution to fermentable sugars.

### CROSS-REFERENCE TO RELATED APPLICATION

This application is United States National Stage application filed under 35 U.S.C. § 371 of International Patent Application No. PCT/US23/16586 filed on Mar. 28, 2023 entitled System And Method To Create A Water-Soluble Mixture Of Oligosaccharides For Facile Conversion To Fermentable Sugars, which claims the priority benefit of U.S. Provisional Ser. No. 63/304,027 entitled System and Method to Create a Water-Soluble Mixture of Oligosaccharides for Facile Conversion to Fermentable Sugars filed Jan. 28, 2022, the entire contents of which are incorporated by reference herein for all purposes.

### FIELD OF THE INVENTION

The present invention relates generally to system and method for and synthesis of a water-soluble mixture of oligosaccharides from cellulosic materials from a solid-solid reaction. More particularly, the present invention relates to certain new and useful advances in systems and methods to achieve reaction conditions that can be used to form a mixture of oligosaccharides under mild conditions which in combination with enzymatic hydrolysis creates an efficient way to reach fermentable simple sugars.

### BACKGROUND

Cellulose is an organic compound with a general formula (C₆HioO₅)ₙ, a polysaccharide consisting of a linear chain of several hundred to many thousands of β(1,4) linked D-glucose units, joined by an oxygen (ether) linkage to form long molecular chains that are essentially linear. These linkages cause the cellulose to have a high crystallinity and thus a low accessibility to enzymes or acid catalysts. This phenomenon is known as recalcitrance.

Cellulose is an important structural component of the primary cell wall of green plants, many forms of algae and the oomycetes. It occurs in close proximity to hemicellulose and lignin, which together comprise the major components of plant fiber cells. In addition, some species of bacteria secrete it to form biofilms. Naturally formed by plants, cellulose is the most abundant organic polymer on Earth.

Enzymes, which perform hydrolyzing function, are a specific type of catalyst, like liquid or solid acids. Hydrolysis, meaning water-cleavage is a reaction involving the breaking of a bond in a molecule using water. Hydrolysis of cellulose yields a mixture of simple reducing sugars, mainly glucose. These hydrolysis products can be converted to ethyl alcohol which can be used as a liquid fuel to replace petroleum, and results in more complete and cleaner combustion, they may also serve as fuel or intermediates in pathways to other fuels. In addition, products of hydrolysis can also be used to manufacture various organic chemicals presently produced from petroleum. In terms of available energy, expressed as the heat of combustion of cellulose or of the glucose product theoretically obtainable therefrom, a pound of cellulose is equivalent to approximately 0.35 lbs. of gasoline or other fuels.

On earth, it has been estimated that roughly 6.45×10¹¹ tons of carbon are fixed and deposited every year by photosynthesis, out of which half appears in the form of cellulose. In addition, it has been estimated that about ¾ of the approximate of the biomass generated on cultivated lands and grasslands currently contribute to waste production. The utilization of such waste materials for developing alternative sources of fuels, chemicals and other useful products has long been desired. However, attempts to hydrolyze cellulose have not yet succeeded in providing an economically viable method for producing sugars, due primarily to the crystalline structure of cellulose and the presence of lignin therein. The sheer magnitude of this potential source dictates the necessity of improving the methods and systems for cellulose utilization.

Furthermore, in known processes and methods the chemical or thermal stress on the macromolecules, particularly when processing extremely viscous, highly substituted products, is so intense that during conversion macromolecules may be decomposed in the form of a chain scission, which is noticeable in particular by the large decrease in viscosity compared to the starting products. Also, the surfaces of the products treated by the preliminary embrittlement or drying steps become rough. Furthermore, a common feature of known processes is the large amount of energy expended in converting cellulose derivatives after the preliminary drying, embrittlement or compaction.

Current cellulosic ethanol is made by taking water insoluble cellulose, slurrying it in large tanks and stir it with cellulase for weeks to months. Because cellulose is linear, beta-linked, highly crystalline material the enzymes slowly etch the surface and break off glucose units eventually bringing all the sugars into solution. Since the cellulose is solid, the only interactions that can occur with the enzymes occur at the surface. This process is generally very slow, expensive and inefficient. It typically also requires a pretreatment with harsh chemicals in order for the reaction to occur. Enzymes such as amylase are able to digest water soluble starches. Cellulases are able to perform solid cellulose digestions but are extremely slow and expensive. The enzymatic breakdown of insoluble cellulose requires a cocktail of enzymes for first digesting the larger solid cellulosic polymers and then converting the oligosaccharides into simple sugars. Beta glucosidases are well known to hydrolyze water soluble oligosaccharides and starches into easily fermentable di-saccharides and mono-saccharides. This type of enzyme is much faster and cost effective than cellulase but requires the cellulose to be broken into much smaller water-soluble units.

What is needed is a system and method for and synthesis of a water-soluble mixture of oligosaccharides and products produced therefrom cellulosic materials that obviates the above-recited drawbacks.

### SUMMARY OF THE INVENTION

The following summary of the invention is provided in order to ensure a basic understanding of some aspects and features of the invention. This summary is not an extensive overview of the invention and as such it is not intended to particularly identify key or critical elements of the invention or to delineate the scope of the invention. Its sole purpose is to present some concepts of the invention in a simplified form as a prelude to the more detailed description that is presented following.

To achieve the foregoing and other aspects and in accordance with the purpose of the invention, an automatic or manual tunable process to create a mixture of water-soluble oligosaccharides that can easily be enzymatically converted to simple, fermentable sugars is provided.

The process converts the cellulose into a product mixture that is fully water soluble. The mixture contains linear glucose oligomers 6 units or less that are water soluble, monosaccharides, and larger repolymerized products that are alpha linked and or branched that increases their solubility.

Advantages of this repolymerization are that it creates a more stable product as the larger molecules are less susceptible to the temperature/pressures of the reaction, allows for all the product mixture to be water soluble to react with enzymes much faster than if solids were present because molecular contact is much easier, allows for the use of the much cheaper enzyme amylase to be used in combination to cellulase instead of exclusively cellulase which is more expensive.

In an embodiment, a solid-solid reaction cellulose reaction in solid phase between a cellulosic feedstock and a catalyst (e.g., clay), and the system then recombines the cellulose but recombines. This process, together with the enzymatic digestion described herein, makes non-soluble cellulose soluble, and thus easier for enzyme degradation, and the recombined portions can be digested with amylases and cellulase instead of just cellulases providing a cost savings and increased yield.

In one embodiment of the present invention, the invention presents a method for the formation of a mixture of water-soluble saccharides and oligosaccharides from lignocellulosic material using a solid-solid reaction as described herein. This mixture is created with the purpose of preparing the output for facile enzymatic digestion to fermentable sugars.

The solid-solid reaction, in embodiments, converts cellulose to sugar using at least a set of rollers or grinding elements as to achieve optimized sugar output from a feedstock of biomass (e.g., cellulose containing material).

The rollers are provided in connection with a braking assembly and geared specifically to control revolutions per minute (“RPM”) on a per grinder basis with a high degree of specificity (˜0.001 mph) to achieve high durability and high output. Further, having the grinders rotating at non-analogous RPMs achieves greater micro-mixing on the solid-solid reaction because this portion of the reaction does not rely on pressure, but rather, it relies on simultaneous grinding with the pressure already provided. The plurality of sensors and operating unit (e.g., PLC) control and optimize micro-mixing via variable speed control to each of the grinders in a non-analogous fashion. However, analogous RPM may also be used.

In embodiments, the methods comprise providing a portion of cellulosic biomass that may be pretreated to optimize particle size, and using the grinders operating at non-analogous RPM, micro-mixing to induce a solid-solid chemical reaction by applying impact forces with shearing forces so that the contract stress is applied to the biomass to perform the reaction.

The present system utilizes mixing generally, but specifically micro-mixing to maximize reaction points in the cellulose whilst ensuring the time that the feedstock has to react is increased. Micro-mixing improves reaction site and catalyst interaction and optimizes energetic performance. The rollers are able to be set such that they are fully adjustable, so that mechanical, temperate, atmospheric, and chemical reaction parameters are controlled. This is to ensure ideal conditions to achieve reaction speed and process efficiency.

The products formed by this reaction are then enzymatically digestion as described herein. The process makes non-soluble cellulose soluble, and thus easier for enzyme degradation. The recombined portions can be digested with amylases and with cellulases instead of just cellulases providing cost savings and increased yield.

Other features, advantages, and aspects of the present invention will become more apparent and be more readily understood from the following detailed description, which should be read in conjunction with the accompanying drawings.

### BRIEF DESCRIPTION OF THE DRAWINGS

The present invention is best understood by reference to the detailed Figures and description set forth herein.

FIG. 1 is a perspective front view of the crusher assembly used within cellulose to sugar a mill in accordance with one embodiment of the present invention;

FIG. 2 is a flow diagram of the process to for the water soluble oligosaccharides in accordance with embodiments of the present invention'

FIG. 3 is a side schematic view of a mill used in the cellulose to sugar process in accordance with one embodiment of the present invention;

FIG. 4 is stepwise method for enzymatic conversion of cellulosic materials to fermentable sugars; and

FIG. 5 schematic view of a system for converting cellulose to fermentable sugars.

### DETAILED DESCRIPTION OF EMBODIMENTS

Before explaining at least one embodiment of the presently disclosed and/or claimed inventive concept(s) in detail, it is to be understood that the presently disclosed and/or claimed inventive concept(s) is not limited in its application to the details of construction and the arrangement of the components or steps or methodologies set forth in the following description or illustrated in the drawings. The presently disclosed and/or claimed inventive concept(s) is capable of other embodiments or of being practiced or carried out in various ways. Also, it is to be understood that the phraseology and terminology employed herein is for the purpose of description and should not be regarded as limiting.

Unless otherwise defined herein, technical terms used in connection with the presently disclosed and/or claimed inventive concept(s) shall have the meanings that are commonly understood by those of ordinary skill in the art. Further, unless otherwise required by context, singular terms shall include pluralities and plural terms shall include the singular.

All patents, published patent applications, and non-patent publications mentioned in the specification are indicative of the level of skill of those skilled in the art to which this presently disclosed and/or claimed inventive concept(s) pertains. All patents, published patent applications, and non-patent publications referenced in any portion of this application are herein expressly incorporated by reference in their entirety to the same extent as if each individual patent or publication was specifically and individually indicated to be incorporated by reference.

All of the articles and/or methods disclosed herein can be made and executed without undue experimentation in light of the present disclosure. While the articles and methods of the presently disclosed and/or claimed inventive concept(s) have been described in terms of preferred embodiments, it will be apparent to those of skill in the art that variations may be applied to the articles and/or methods and in the steps or in the sequence of steps of the method described herein without departing from the concept, spirit and scope of the presently disclosed and/or claimed inventive concept(s). All such similar substitutes and modifications apparent to those skilled in the art are deemed to be within the spirit, scope and concept of the presently disclosed and/or claimed inventive concept(s).

As utilized in accordance with the present disclosure, the following terms, unless otherwise indicated, shall be understood to have the following meanings.

The use of the word “a” or “an” when used in conjunction with the term “comprising” in may mean “one,” but it is also consistent with the meaning of “one or more,” “at least one,” and “one or more than one.” The use of the term “or” is used to mean “and/or” unless explicitly indicated to refer to alternatives only or the alternatives are mutually exclusive, although the disclosure supports a definition that refers to only alternatives and “and/or.” Throughout this application, the term “about” is used to indicate that a value includes the inherent variation of error for the device, the method being employed to determine the value, or the variation that exists among the study subjects. For example, but not by way of limitation, when the term “about” is utilized, the designated value may vary by plus or minus twelve percent, or eleven percent, or ten percent, or nine percent, or eight percent, or seven percent, or six percent, or five percent, or four percent, or three percent, or two percent, or one percent. The use of the term “at least one” will be understood to include one as well as any quantity more than one, including but not limited to, 2, 3, 4, 5, 10, 15, 20, 30, 40, 50, 100, etc. The term “at least one” may extend up to 100 or 1000 or more, depending on the term to which it is attached; in addition, the quantities of 100/1000 are not to be considered limiting, as higher limits may also produce satisfactory results. In addition, the use of the term “at least one of X, Y and Z” will be understood to include X alone, Y alone, and Z alone, as well as any combination of X, Y and Z. The use of ordinal number terminology (i.e., “first”, “second”, “third”, “fourth”, etc.) is solely for the purpose of differentiating between two or more items and is not meant to imply any sequence or order or importance to one item over another or any order of addition, for example.

As used herein, the words “comprising” (and any form of comprising, such as “comprise” and “comprises”), “having” (and any form of having, such as “have” and “has”), “including” (and any form of including, such as “includes” and “include”) or “containing” (and any form of containing, such as “contains” and “contain”) are inclusive or open-ended and do not exclude additional, unrecited elements or method steps.

The term “or combinations thereof” as used herein refers to all permutations and combinations of the listed items preceding the term. For example, “A, B, C, or combinations thereof” is intended to include at least one of: A, B, C, AB, AC, BC, or ABC and, if order is important in a particular context, also BA, CA, CB, CBA, BCA, ACB, BAC, or CAB. Continuing with this example, expressly included are combinations that contain repeats of one or more item or term, such as BB, AAA, MB, BBC, AAABCCCC, CBBAAA, CABABB, and so forth. The skilled artisan will understand that typically there is no limit on the number of items or terms in any combination, unless otherwise apparent from the context.

Specific configurations and arrangements of the platform, discussed above regarding the accompanying drawing, are for illustrative purposes only. Other configurations and arrangements that are within the purview of a skilled artisan can be made, used, or sold without departing from the spirit and scope of the platform. For example, a reference to “an element” is a reference to one or more elements and includes equivalents thereof known to those skilled in the art. All conjunctions used are to be understood in the most inclusive sense possible. Thus, the word “or” should be understood as having the definition of a logical “or” rather than that of a logical “exclusive or” unless the context clearly necessitates otherwise. Structures described herein are to be understood also to refer to functional equivalents of such structures.

While the present platform has been described in connection with what are presently considered to be the most practical and preferred embodiments, it is to be understood that the present platform is not limited to these herein disclosed embodiments. Rather, the present platform is intended to mobile phone the various modifications and equivalent arrangements included within the spirit and scope of the appended claims.

Although specific features of various embodiments of the platform may be shown in some drawings and not in others, this is for convenience only. In accordance with the principles of the platform, the feature(s) of one drawing may be combined with any or all the features in any of the other drawings. The words “including,” “comprising,” “having,” and “with” as used herein are to be interpreted broadly and comprehensively and are not limited to any physical interconnection. Moreover, any embodiments disclosed herein are not to be interpreted as the only possible embodiments. Rather, modifications and other embodiments are intended to be included within the scope of the appended claims.

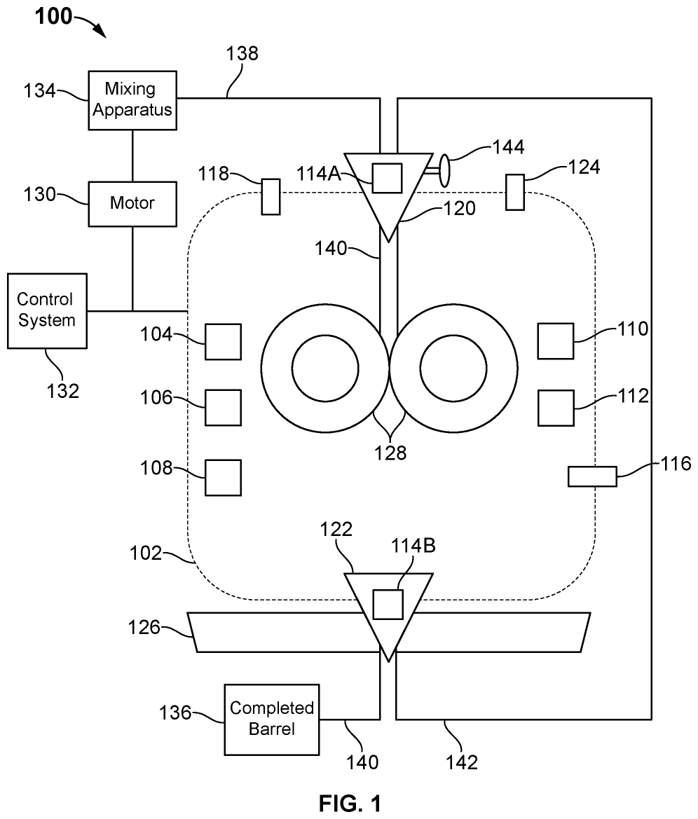

In embodiments, Lignocellulosic material is chemically reacted by “Cellulose to Sugar Technology” using a solid-solid reaction as shown by use of a crusher assembly e.g., roller mill, for example, shown in FIGS. 1-3 in either a batch or continuous process.

Referring now to FIG. 1, a perspective front view of an embodiment showing a system namely a mill, that can be used in the cellulose to sugar process in accordance with one embodiment of the present invention, is presented generally at reference numeral 100. This embodiment 100 illustrates the functional components of the mill 100 in accordance with one embodiment of the present invention. The various components of the mill 100 and their role in the cellulose to sugar process will be further described below in relation to FIGS. 1-3. The mill 100 comprises a reactor chamber 102 with a plurality of control components. In one embodiment, the plurality of control components comprises an inlet hopper 120, a crusher assembly 128, an outlet hopper 122, a sensor assembly, a steam inlet 118, and a carbon dioxide inlet 124.

Still referring to FIG. 1, a control system 132 is coupled to a drive assembly 130 and both are coupled to the reactor chamber 102. In one embodiment, the drive assembly 130 includes a motor. In one embodiment, the motor 130 is powered via a power supply. By being coupled to the reactor chamber 102, the control assembly 132 is able to communicate and receive information from the various sensors 104-112, vacuum pump 116, heater 126, crusher assembly 128, steam inlet 118, carbon dioxide (CO²) inlet 124 and detectors 114A-114B. Through its interconnectivity, the control assembly 132 allows for real time monitoring, analyzing, and adjusting to ensure that the process is optimized. The foregoing is further discussed herein when describing the other components of the device.

Referring still to FIG. 1, the crusher assembly 128 is configured to induce a chemical reaction in solid phase between the feedstock and the catalyst (e.g., clay). In one embodiment, the crusher assembly 128 may be a single set of approximately smooth rollers (e.g. rounded), but any shape roller may be used so long as it induces appropriate pressure. In another embodiment, the crusher assembly 128 may be a set of intermeshing rollers in the form of gears with high hardness. In some embodiments, the crusher assembly 128 may be any mechanism to compress the solids at very high pressure. The crusher assembly 128 is configured to compress or push together the solids at very high pressure and at a predetermined temperature which aids a solid-solid molecular reaction between the feedstock and the hydrous clay to produce or synthesize sugar utilizing a feedstock. In one embodiment, the solids include, but are not limited to, a lignocellulosic biomass and solid acids. In one embodiment, the ratio of the biomass to the solid acid may be, but is not limited to, 1:0.1-10 kg:kg. In one embodiment, the solid acids may be, but are not limited to, kaolin, bentonite, and montmorillonite or any solid acid existing now or in the future.

Still referring to FIG. 1, the drive assembly 130 and control assembly 132 are also coupled to the mixing apparatus 134, which is where the feedstock and catalyst are mixed; once mixed, the material is sent to the inlet hopper 120 via the feed line 138. Once inside the inlet hopper 120, the detector 114A together with any other necessary sensors or detectors analyzes the matter and calculates information that will be useful in the process such as protein content, cellulose, starch, and monomeric sugar, water, lignin, ash, oil, and mechanical properties. In one embodiment, the detector (114A and 114B) is a NIR detector but may be any detector or sensor that analyzes compounds and materials in a mixture. This information will be used to analyze the material to ensure the process performs at the optimal level to ensure consistency and the best yield. In one embodiment, readings from the detector 114A can be utilized by the control assembly 132 to make adjustments to the speed of the crusher assembly 128 to ensure the process is optimized. Once the material is analyzed inside the inlet hopper 120, then the feed valve 144 will be used to open the inlet hopper 120 so that the material may pass from the inlet hopper 120 down into the feed guide 140, which will guide the material down between the crusher assembly 128 located within the reactor chamber 102. As previously discussed, the crusher assembly 128 is powered via the drive assembly 130 and control assembly 132 that are coupled to the reactor chamber 102. In one embodiment, the crusher assembly 128 and the drive assembly 130 are connected via a drive shaft. Once the process is completed, the material exits the reactor chamber 102 via the outlet hopper 122. Once in the outlet hopper 122, the detector 114A and 114B together with any other necessary sensors or detectors analyzes the material to determine whether or not it must be passed through the mill 100 again. If it is determined that the material must be ran through again, then the material will be sent via the return feed line 142 back to the inlet hopper 120, where the detector 114A will analyze the material again, whilst determining the adjustments which must be made to the device in order to reprocess the material. Once the process is completed and the material is no longer required to be run through the crusher assembly 128, then it will be sent to the completed collection device 136 via the exit feed line 140. In one embodiment, an outlet valve could be provided at the feed guide or line 140 to control the flow of the material. In one embodiment, a tight seal is provided to the feed lines 140 and 142 to prevent leakages of the material. It is important to note that more than one crusher assembly 128 may be used in the chamber 102.

Still referring to FIG. 1, the inlet hopper 120 and the outlet hopper 122 are coupled to the reactor chamber 102 and are used to introduce the material into the collection device 102 and to evacuate the material out of the collection device 102, respectively. To open and close the inlet hopper 120 so that the material may enter the reactor chamber 102, a feed valve 144 is used. In the present embodiment, the inlet hopper 120 and outlet hopper 122 are operated based upon an atmospheric control system that regulates pressure in the reactor chamber 102 to enhance conveyance of materials in the system. In other embodiments, the inlet hopper 120 and outlet hopper 122 may be controlled via electronic systems and coupled with the control assembly 132.

Still referring to FIG. 1, a control assembly 132 is coupled to the drive assembly 130 that is further coupled to the crusher assembly 128 which is further coupled to the reactor chamber 102. The drive assembly 130 must provide enough power and torque required to turn the crusher assembly 128 at a predetermined or optimal revolutions per minute and be able to change speeds and power outputs over time. In embodiments, each of the rollers of the crusher assembly 128 may turn at different RPMs in order to optimize the reaction. In one embodiment, the control assembly 132 is a processor that reads the sensors 104-112 and automatically responds to predefined parameters. Real time measurements will allow for real time adjustments to ensure the crusher assembly 128 operates in the optimal manner. As an example, the drive assembly 130 and control assembly 132 may alter the revolutions per minute as needed to adjust the torque and power of the crusher assembly 128 based upon sugar production and responses from the parameter monitoring. In another example, if the temperature sensor 106 sends a reading to the control assembly 132 that the temperature is outside of a predetermined range, then the control assembly 132 will send a corresponding signal to the heater 126 to heat the reactor chamber 102.

Still referring to FIG. 1, the mill 100 further comprises a sensor assembly. In embodiments, the sensor assembly comprises various sensors 104-112, which are coupled to the interior of the reactor mill 102, which include a pH sensor 104, temperature sensor 106, oxygen sensor 108, moisture sensor 110 and pressure sensor 112, all of which are described herein in further detail. All of the sensors 104-112 will also be coupled to the control assembly 132 in order to communicate to the other systems and devices that may be coupled to the reactor chamber 102 to ensure the production of cellulose is at its optimal level, all of which are further described herein. The pH sensor 104 is coupled to the reactor chamber 102 and aids in measuring the effective acidity of the reaction environment. The pH sensor is configured to measure hydrogen ion concentration of the solution which aids in establishing the actual acidity of each site and the number of acid sites. Because hydrolysis is catalyzed by acid sites on the catalyst, a lower pH indicates more acid sites, increasing the changes for hydrolysis to occur. In addition, monitoring the pH levels and assuring certain levels are met will also affect fermentation and/or conversion of the materials loaded into the reactor chamber 102 process. The temperature sensor 106 may be coupled to the reactor chamber 102 and is used to monitor the frictional heat temperature within the reactor chamber 102 to ensure that a high enough temperature is reached to activate the hydrolysis reaction occurring between water and cellulose to make sugar; at the same time, this temperature must also be low enough to avoid reactions that would cause the sugar to degrade.

Still referring to FIG. 1, the oxygen sensor 108 may be coupled to the reaction chamber 102 and is used to monitor oxygen levels within the reaction chamber 102. Because oxygen can cause oxidation of sugar products, it must be removed from the reaction chamber 102 before the cellulose to sugar process can be completed. To accomplish the foregoing, the oxygen sensor 108 works in conjunction with the vacuum pump 116, which is also coupled to the reaction chamber 102, such that if the oxygen sensor 108 detects any oxygen within the reaction chamber 102, the oxygen sensor 108 will communicate to the vacuum pump 116 via the control assembly 132, which both the oxygen sensor 108 and vacuum pump 116 are also coupled to, to release such oxygen out of the reaction chamber 102. These sensors may be referred to herein atmospheric equilibrium sensor/devices work in conjunction with other to optimize the conditions in the mill 100.

Still referring to FIG. 1, the oxygen sensor 108 also works in conjunction with the CO₂ inlet 124, which is also coupled to the reaction chamber 102 as well as the control assembly 132. Thus, if the oxygen sensor 108 detects oxygen in the reaction chamber 102 and communicates to the vacuum outlet 116 to release the same via the control assembly 132, the carbon dioxide inlet 124 will automatically add protective inert carbon dioxide gas to the reaction chamber 102 in order to maintain a positive CO₂ pressure within the reaction chamber 102.

Still referring to FIG. 1, a moisture sensor 110 is coupled to the reaction chamber 102 and is used to monitor the amount of moisture within the reaction chamber 102. In one embodiment of the present invention, moisture acts as a reactant to produce sugar during the cellulose to sugar process and is consumed by the reaction. As sugar is produced, the moisture levels in the reaction chamber 102 drops and the moisture localizes to hydrate the more hydroscopic monomeric sugars being produced. Therefore, the moisture sensor 110 is important in the present embodiment to ensure that the moisture levels in the reaction chamber 102 remain at the optimal level for the best reaction. In the present embodiment, the moisture levels may be greater than 0.00% but less than 50% by mass. To ensure the foregoing moisture levels are maintained, a steam inlet 118 is also coupled to the reaction chamber 102 and is used to disperse additional steam into the reaction chamber 102, such that the moisture sensor 110 may communicate via the control assembly 132 with the steam inlet 118 to disperse additional steam into the reaction chamber 102.

Still referring to FIG. 1, spectrum detectors 114A-114B together with any other necessary sensors or detectors are coupled to the inlet hopper 120 and outlet hopper 122, respectively, and may be used to analyze the compositions as they pass through the hoppers. The detectors 114A-114B together with any other necessary sensors or detectors will provide data on protein content, cellulose, starch, water, monomeric sugar, lignin, ash and oil. In future embodiments, algorithms may be used to automate responses through the control assembly 134. In one embodiment, the detector 114B coupled to the outlet hopper 122 will determine whether or not the material must be passed through the device again; if the spectrum detector 114B determines it must be passed through again, then the material is returned to the inlet hopper 120 via the return feed line 142. In one embodiment, a feed pump may be provided at the feed line 142 for returning the material to the inlet hopper 120.

Still referring to FIG. 1, a pressure sensor 112 is coupled to the reaction chamber 102 and is used to monitor the pressure within the reaction chamber 102. The pressure required to induce hydrolysis is created by the crusher assembly 128 within the reaction chamber 102, but the pressure in the reaction chamber 102 must be monitored as the pressure may increase or decrease with the changing temperature, requiring CO² to be added to the reaction chamber 102 via the CO² inlet 124 in order to maintain the optimal pressure for the reaction.

Still referring to FIG. 1, a heater 126 is coupled to the base of the reaction chamber 102. While the heat required for the cellulose to sugar process to occur mostly comes from the friction created within the reaction chamber 102 during the process, the initial heating of the reaction chamber 102 may be carried out using the heater 126. In other optional embodiments, the cooling process may be carried out using fans along with heat sinks coupled to the reaction chamber or the gears or rollers themselves and controlled via the control assembly 132. The crusher assembly and the rollers may also be temperature controlled by either internal heating or cooling elements or external heating and cooling elements.

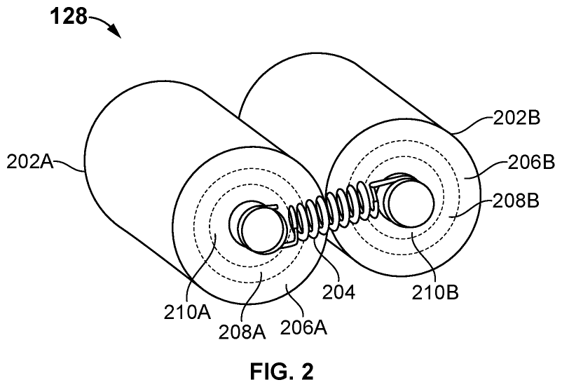

Referring to FIG. 2, a perspective front view of the crusher assembly 128 used within the mill 100 is presented. The crusher assembly 128 comprises two smooth rollers 202A-202B that are pressed together using a spring 204, but any device that is able to produce high pressure may be used, for example, hydraulic pistons, screws and any other mechanism to induce pressure. As discussed herein with reference to FIG. 1, the crusher assembly 128 is turned at a rate by the drive assembly 130, which uses the readings from all of the various sensors 104-112 to determine the optimal rate. The smooth roller is made of materials that have excellent wear properties to endure long run times at high pressures and in embodiments, are manufactured using various materials having differing hardness.

Each of the rollers 202A and 202B may be formed of material having various degrees of hardness (i.e., layers formed of different materials). In exemplar embodiments, the rollers 202A and 202B have three tiers 206A and 206B, 208A and 208B, and 210A and 210B. The outer tier 206A and 206B have, relatively, the highest hardness. The inner tier 210A and 210B has the least or lowest hardness and the middle tier 208A and 208B have a hardness that falls in between the outer tier 206A and 206B and inner tier 210A and 210B. In operation, having the rollers 202A and 202B being formed of varying hardness optimizes the reaction because it increases micro-reactions of the materials. The outer tier 206A and 206B having high hardness ensures that the pressure on the materials remains high and having the middle tier of differing hardness (or softer hardness) ensures that the energy is not lost due to compressive forces in the outer tier being too high and to prevent compression of the roller material. By varying the pressure over the depth of the roller, we can tune the surface and therefore the reaction space and energetic efficiency. The number, thickness, aspect ratio, length, diameter, and material type of layers may be optimized depending upon the feedstocks and such factors influence properties of hardness, toughness, compressive strength, and wear resistance.

In one embodiment, the rollers 202A and 202B may be made with gear teeth because they have hard surfaces, which induces beneficial compressive residual stresses that effectively lower the load stress, in other embodiments, the rollers may be made of strong metals and alloys, tungsten carbide, diamond, plastics, ceramics and composite materials and the like. In an embodiment, the axels that utilize motive force to spin the rollers may be supplied by an adequate supply of cool, clean and dry lubricant that has adequate viscosity and a high pressure-viscosity coefficient may also be used to help prevent pitting, a fatigue phenomenon that occurs when a fatigue crack initiates either at the surface of the gear tooth or at a small depth below the surface. In one embodiment, the bearings could be, but is not limited to, ball bearings. The teeth on the individual gears 202A and 202B must also be designed for most efficient wear properties as well as reaction efficiency in regard to contact area and pressure. While only two sets of rollers are shown, there may be an infinite number of rollers in series. Rollers and gears are composed of surfaces for reaction purposes and contact with feed mixture whereas surfaces of the roller or gear support can compose of surfaces that reduce friction and enhance wear resistance and drive surfaces will be enhanced for the use of pulleys, belts, sprockets, chains, couplings and direct drive attachments.

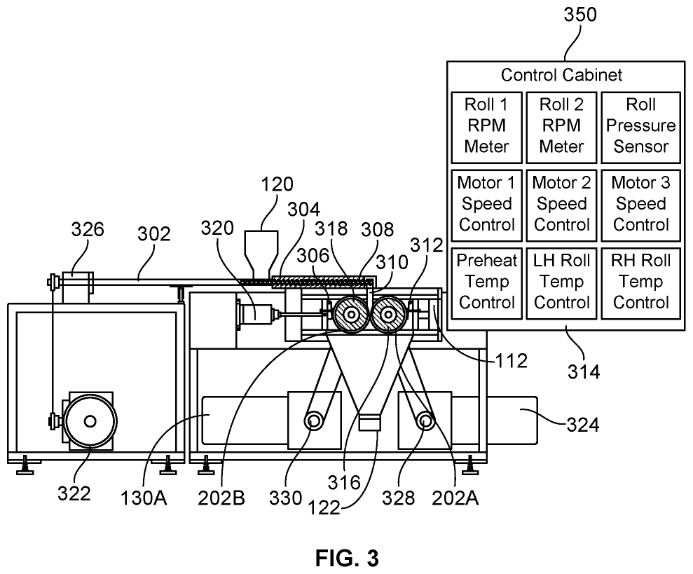

An exemplary embodiments of a system that produces sugars according the above reaction is shown in FIG. 3. Referring now to FIG. 3, a side schematic view of a mill 100 used in the cellulose to sugar process in accordance with one embodiment of the present invention is shown at 300. As shown a biomass hopper 120 is provided at the top of the system and configured to accept biomass raw material whether it is pre-treated or not pre-treated. A conveying screw tube 302 is in communication with the bottom of the hopper 120 and configured to provide the raw material biomass into the system and to separate the raw material to so that the flow is even, constant and congruent to prevent clogs. The conveying screw tube 302 is connected to a motor screw conveyor drive 322 through gearbox 326 to provide power and to the conveying screw 308 in the conveying screw tube 302. An incoming product heater 304 surrounds the incoming conveying screw tube 302 and is configured to provide a predetermined heat to the biomass as it proceeds through the screw tube 302. The conveying screw 308 is provided inside of the conveying screw tube 302 to move raw material down the tube into in the reaction zone via a drop chute 310. The drop chute 310 is in communication and connected with the conveying screw tube 302 to drop the biomass into the reaction zone (e.g., the crusher assembly).

Still referring to FIG. 3, the crusher assembly, a hydraulic cylinder is connected to a cylinder pushrod to drive the crusher assembly which comprises the two rollers 202A-202B that are pressed together to induce a reaction in the biomass and a catalyst to produce sugars. The two rollers 202A-202B are positioned in an internal compartment that also houses left roller scraper 306 and right roller scraper 312. Each of the roller scrapers are in contact with at least one side of the rollers and are configured to remove particulate from each roller as they are driven. In this way, very little raw material is lost and further, the reactions are optimized because the opposite side of the wheel that is performing the reactions is clean and smooth. The particulate that is scraped off of the roller into the outlet chute hopper in communication with the internal compartment and configured to eject the raw materials.

Motor belts 328 and 330 are provided to provide motive force.

With further reference to FIG. 3, two pressure sensors are employed on each side of the internal compartments and are configured to ensure optimal pressure throughout the reaction zone. Each of the sensors are in communication either wirelessly or via wire to the control cabinet 350 in which various other meters and control toggles programmable logic controllers, and circuits are located, for example, roller RPM meter one, roller RPM meter two, roll pressure sensor, motor speed control, pre-heater temperature control, first roll temperature control, and second temperature control.

Raw material that is processed via the rollers 202A and 202B are then released into a product discharge chute 122. The product discharge chute may also employ sensors and sifting mechanisms to provide the optimal products as an output and re-introduce non-optimal products back into the system for processing.

Multiple motors in gearboxes may be employed to provide motive force to the system each of which can be powered in any type of way. Hydraulic motors 324 may be provided to power the rollers, whereas motor 322 may be provided to power the screw conveyor drive 322. Each of the motors may be in communication with control cabinet 350 and each of the sensors provided therein at 314.

Further, the system is tunable and different reaction conditions lead to different product distributions. While the end target of the of the cellulose to sugar process is fermentable sugars, typically consisting of mono-saccharides, in embodiments, the method described herein uses has increased efficiency using milder and faster conditions to form a greater percentage of water soluble oligosaccharides that are both branched and linear, and enzymatically hydrolyze them to fermentable sugars with a fast working and inexpensive enzyme such as amylase and/or cellulase.

In embodiments, several conditions of the feedstock and catalyst are optimized. The moisture of lignocellulosic feed stock is milled with a hammer mill, or other type of mill to a small particle size (e.g., 50-600 microns). The material may also have an optimized moisture content (0.5-25%). The methods may also utilize a ratio of 2:1 (0.1:1-5:1) catalyst to feedstock with the solid acid catalyst being for example kaolin, though other catalysts may be utilized. The catalyst moisture level is also optimized in the method, in the range of 1.0-25.0% moisture for example. The lignocellulosic feedstock and catalyst may be two solids and are physically mixed to have each component evenly distributed. This mixture can be then reacted in a batch reactor in the form of a ball mill or the hammer or roller mill shown in FIG. 1. In either reactor system, the mild acid catalyst activates water molecules in the material which then hydrolytically cleave the ether linkages in cellulose and hemicellulose.

In embodiments, there are several variables that may be optimized in order for the material to be processed correctly. In batch mode, the temperature, moisture levels, reaction time, and configuration of the ball mill are all optimized. In the continuous process, reaction chamber temperature (50-160° C.), pressure achieved at the reaction site (25,000-125,000 psi).

In operation, the system shown or other similar systems, cellulose degradation and repolymerization occurs and the process uses a mild solid acid catalyst to depolymerize cellulose and hemicellulose. In non-aqueous conditions condensation certain reactions take place during use of the system. Cellulose is beta linked glucose units, and the beta sheets degrade to beta linked oligomers. Upon repolymerization, alpha and beta products are formed from esterification. Due to the way in which the cellulose is treated, the hydrolysis and condensation reaction between glucose units of cellulose are in a state of equilibrium that can be driven by reaction conditions and proximity of the molecules. While cellulose is a linear, beta linked polymer, the reaction breaks the ester bond by hydrolysis. This alcohol can reform with other monomers or oligomers by condensation in a linear, branched, alpha or beta orientation. Aqueous degradation form beta linked oligomers whereas the system forms traces of alpha and beta oligomers as a result of the reaction.

In embodiments, the hemicellulose and cellulose are hydrolyzed into sugars of different chain lengths including but not limited to mono, di, tri and oligosaccharides, all referred to as sugars. As the cellulose and hemi cellulose are broken down into simpler sugars, they become water soluble. This process breaks down the water insoluble cellulose and hemicellulose polymers into smaller water-soluble units. The process also leads to some recombination of sugar units to larger molecules. These larger molecules are both alpha linked and beta linked as well as branched which increases its water-solubility. Glucose monomers decompose at much milder conditions of temperature and pressure than cellulose. After monomers or small oligomers repolymerization to alpha linked and/or branched oligomers, a larger more stable molecule is formed. This helps enable the reaction to be driven to completion without the decomposition of the desired product of fermentable sugars. This repolymerized product is now much more water soluble than cellulose.

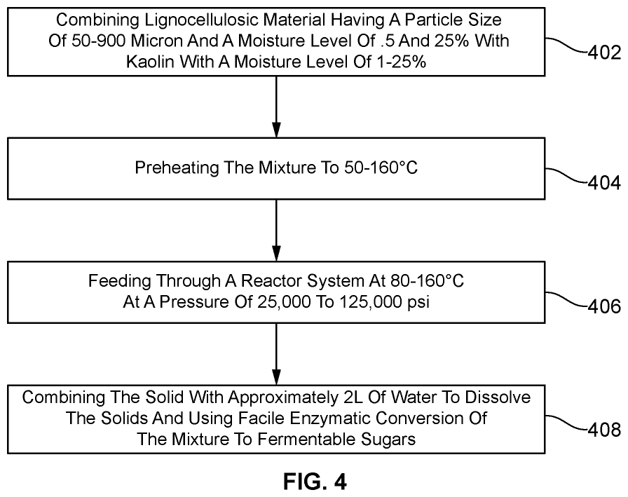

Once the reaction is complete, the cellulose and hemicellulose are broken down and recombined into, water-soluble sugar components through hydrolysis and repolymerization (condensation). The material is dissolved in water via the separator as described in FIG. 4, certain enzymes described herein area convert the larger molecules into smaller, fermentable sugars. The current standard method for breaking down cellulose into fermentable sugars requires a significantly amount of time and cellulase as cellulose is not water soluble and the enzyme slowly etches the surface of the solid and can take weeks or months to fully convert. The process herein creates fully water soluble mixture of broken down cellulose and hemicellulose that can be used with a combination of amylase and cellulase which is more cost effective than cellulase alone and is able to quickly convert the oligo-saccharides and alpha linked sugar units to smaller fermentable sugars within hours.

With reference now to FIG. 4, 100 g of lignocellulosic material with a particle size of 50-900 micron and a moisture level of 4-18% is combined and mixed with 200 g of kaolin with a moisture level of 0.5-25% step 402. This material is preheated to 50-160° C. step 404 and then fed through a reactor system at 80-160° C. at a pressure of 25,000 to 125,000 psi step 406. The solid material product comes out of the reactor system and is combined with approximately 2 L of water with mixing and then separated from the solids (e.g., lignan and the catalyst which may be filtered out or centrifuged out) and the water dissolved solids are the saccharide mixture that is readily available for facile enzymatic conversion to fermentable sugars step 408.

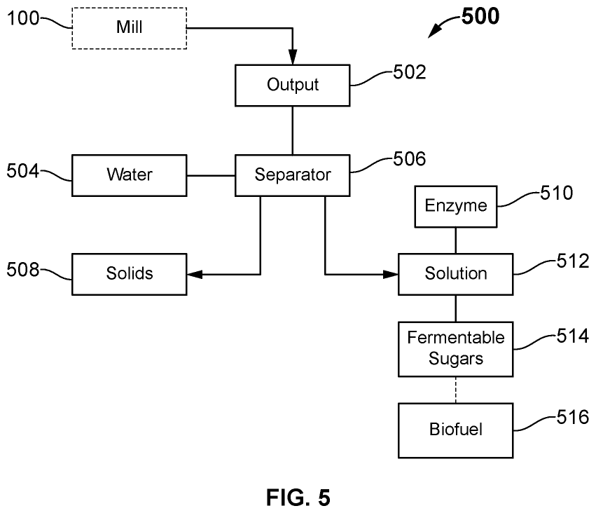

With relation now to FIG. 5 schematic view of a system for converting cellulose to fermentable sugars is shown. A separator 506 is coupled to the output 502 and a water source 504. In operation the separator is configured to separate solids 508 (e.g., lignan and the catalyst) from a solution 512, the latter of which coupled to an enzyme source 518. An output of fermentable sugars is produced, which can then become treated and form, for example, biofuel 516 or bioplastic.

The process converts the cellulose into a product mixture that is fully water soluble. The mixture contains linear glucose oligomers six units or less that are water soluble, monosaccharides, and larger repolymerized products that are alpha linked and or branched that increases their solubility.

The repolymerization creates a more stable product as the larger molecules are less susceptible to the temperature/pressures of the reaction, allows for all the product mixture to be water soluble to react with enzymes much faster than if solids were present because molecular contact is much easier, allows for the use of the much cheaper enzyme amalyse to be used in combination to cellulase instead of exclusively cellulase which is more expensive.

In an embodiment, a solid-solid reaction cellulose reaction in solid phase between a cellulosic feedstock and a catalyst (e.g., clay), and the system then recombines the cellulose but recombines. This process, together with the enzymatic digestion described herein, makes non-soluble cellulose soluble, and thus easier for enzyme degradation, and the recombined portions can be digested with amylases and cellulase instead of just cellulases providing a cost savings and increased yield.

In one embodiment of the present invention, the invention presents a method for the formation of a mixture of water-soluble saccharides and oligosaccharides from lignocellulosic material using a solid-solid reaction as described herein. This mixture is created with the purpose of preparing the output for facile enzymatic digestion to fermentable sugars.

Furthermore, in comparing ratios of alpha to beta linked glucose oligomers, one can have insight to the process used to create sugar products from cellulose using the system. In one embodiments, information is gleaned on the on alpha to beta ratios to “fingerprint” the outputs from the system on a molecular basis.

In this way, devices and methods disclosed herein identify and provide quality control to sugar product derived from cellulosic feedstock. More particularly analytical instrumentation and methodology described herein for sugar product analysis and the trace alternative products give a “fingerprint” in part to what methodology was used to produce said product.

The present invention relates to methods that can be used to identify specifically that mechanocatalytic hydrolysis was used to produce a sugar product.

In one embodiments, the method fingerprint the output compound and molecules utilizes Circular dichroism (CD) (and synchrotron circular dichroism (SCD)) spectroscopy which is a rapid, highly sensitive technique used to investigate structural conformational changes in biomolecules in response to interactions with ligands in solution and in film. It is a chiroptical method and at least one of the interacting molecules must possess optical activity (or chirality). The capabilities of CD and SCD in the characterization of celluloses and lignin polymers in archaeological wood. Cellulose produces a range of spectral characteristics dependent on environment and form; many of the reported transitions occur in the vacuum-ultraviolet region (<180 nm) most conveniently delivered using a synchrotron source. The use of induced CD in which achiral dyes are bound to celluloses to give shifted spectra in the visible region is also discussed, together with its employment to identify the handedness of the chiral twists in nanocrystalline cellulose. This method may be used to identify components of the output of the system.

Another use method of analysis comprises use of mass spectrometry which has high sensitivity and is tolerant of mixtures, and is a natural choice for the analysis of this class of molecules. The characterization of carbohydrates relies upon obtaining the full details of structure from the mass spectrum. Subtle differences due to isomerism or chirality can produce molecules with very different biological activities, making complete structural analysis even more demanding. Mass spectrometry methodologies and technologies for biomolecule analysis continue to rapidly evolve and improve, and these developments have benefited carbohydrate analysis. These developments include approaches for improved ionization, new and improved methods of ion activation, advances in chromatographic separations of carbohydrates, the hybridization of ion mobility and mass spectrometry, and better software for data collection and interpretation. In this way, these method may be used to identify components of the output of the system.

Unique to the system described above, process trace amounts of dehydration products are produced relative to aqueous cellulose deconstruction techniques. In this way, in situ mass spectrometry of glucose decomposition under hydrothermal reactions may be used to identify outputs of the reactions. Understanding on a molecular level the acid-catalyzed decomposition of the sugar monomers from hemicellulose and cellulose (e.g. glucose, xylose), the main constituent of lignocellulosic biomass is very important to increase selectivity and reaction yields in solution. The CAD mass spectrum of protonated d-glucose is characterized by the presence of ionic dehydrated daughter ion (ionic intermediates and products), which may be structurally characterized by their fragmentation patterns. In this way, these methods may be used to identify components of the output of the system.

Another use method of analysis comprises analyzing elemental analysis for forensic determination of product once turned to ethanol. In operation, the method determines of trace elements in fuel ethanol by ICP-MS using direct sample introduction by a microconcentric nebulizer. Normally, products such as biodiesel and bioethanol are mixed with conventional fossil fuels (diesel and gasoline, respectively). Therefore, metals come from the raw product employed for biofuel production (seeds, sugars, etc.) as well as from the production and storage process or even from the added fuels. The determination of the final metal and metalloid concentration in biofuels is a challenging subject because of several reasons. On the one hand, their content is usually low (i.e., from several μg L⁻¹ to mg L⁻¹). Due to this, biofuel analysis through ICP-OES and ICP-MS may be used to In this way, these method may be used to identify components biofuel that is manufactured and formulated using the output sugars of the system.

The table illustrates the composition of oligomer and dehydration products in glucose product and ultra-trace metals analysis of bioethanol from the enzymatic and aqueous processes described herein.

TABLE 1

| | | | | Solids- |
|---|---|---|---|---|
| | | | | Solid |
| | | Enzyme | Aqueous | Reaction |
| Oligomer | Total Oligomer | trace | 0.08% | 0.12% |
| Fraction | | | | |
| | Alpha/Beta | N/A | 0.2 | 0.5 |
| | Ratio | | | |
| Dehydration | Dimethyl- | trace | 0.03% | 0.80% |
| product | furfural | | | |
| Fraction | | | | |
| | Hydroxymethyl- | trace | 0.05% | 0.12% |
| | furfural | | | |
| | Ethoxymethyl- | trace | trace | 0.02% |
| | furfural | | | |
| Metals in EtOH | Si | ultra trace | ultra trace | 0.03 ppt |
| | Al | ultra trace | ultra trace | 0.03 ppt |

Specific configurations and arrangements of the platform, discussed above regarding the accompanying drawing, are for illustrative purposes only. Other configurations and arrangements that are within the purview of a skilled artisan can be made, used, or sold without departing from the spirit and scope of the platform. For example, a reference to “an element” is a reference to one or more elements and includes equivalents thereof known to those skilled in the art. All conjunctions used are to be understood in the most inclusive sense possible. Thus, the word “or” should be understood as having the definition of a logical “or” rather than that of a logical “exclusive or” unless the context clearly necessitates otherwise. Structures described herein are to be understood also to refer to functional equivalents of such structures.

While the present platform has been described in connection with what are presently considered to be the most practical and preferred embodiments, it is to be understood that the present platform is not limited to these herein disclosed embodiments. Rather, the present platform is intended to mobile phone the various modifications and equivalent arrangements included within the spirit and scope of the appended claims.

Although specific features of various embodiments of the platform may be shown in some drawings and not in others, this is for convenience only. In accordance with the principles of the platform, the feature(s) of one drawing may be combined with any or all the features in any of the other drawings. The words “including,” “comprising,” “having,” and “with” as used herein are to be interpreted broadly and comprehensively and are not limited to any physical interconnection. Moreover, any embodiments disclosed herein are not to be interpreted as the only possible embodiments. Rather, modifications and other embodiments are intended to be included within the scope of the appended claims.

1. A system for generating a water-soluble mixture of oligosaccharides for facile conversion to fermentable sugars, the system comprising:
a reaction chamber;
a crusher assembly configured to receive a mixture of a cellulose feedstock and a solid acid catalyst, wherein the crusher assembly is configured to grind the mixture under pressure to induce a solid-solid interaction between the cellulosic feedstock and the solid acid catalyst to induce a chemical reaction to produce a grinded mixture, wherein the crusher assembly comprises rollers;
a separator in communication with a water source and configured to receive water from the water source and receive the grinded mixture, wherein the separator is configured to separate solids from the grinded mixture to generate a solution, wherein the solution is output to a solution chamber, and wherein the solution comprises the oligosaccharides;
a solution chamber configured to receive the solution, wherein the solution chamber is coupled to an enzyme source in which the solution is enzymatically converted to fermentable sugars.

2. The system of claim 1, wherein fermentable sugars are further treated to form biofuel.

3. The system of claim 1, wherein the cellulosic feedstock, after, separation in the grinded mixture is fully water soluble.

4. The system of claim 1, wherein the oligosaccharides produced are linear glucose oligomers six units or less, monosaccharides, and larger repolymerized products that are alpha linked and or branched that increase their solubility.

5. The system of claim 1, wherein the enzymes are a mixture of amylase and cellulase.

6. A method for generating a water-soluble mixture of oligosaccharides for facile conversion to fermentable sugars, the method comprising:
crushing, grinding, or both, a mixture of a cellulose feedstock and a solid acid catalyst, under pressure to induce a solid-solid interaction between the cellulosic feedstock and the solid acid catalyst to induce a chemical reaction to produce a grinded mixture, wherein the crusher assembly comprises rollers;
introducing water to separate the grinded mixture into solids and a solution, wherein the solution comprises the oligosaccharides;
enzymatically converting the solution to fermentable sugars.

7. The method of claim 5, wherein fermentable sugars are treated to form biofuel.

8. The method of claim 5, wherein the cellulosic feedstock, after, separation in the grinded mixture is fully water soluble.

9. The method of claim 5, wherein the oligosaccharides produced are linear glucose oligomers six units or less, monosaccharides, and larger repolymerized products that are alpha linked and or branched that increase their solubility.

10. The system of claim 5, wherein the enzymes are a mixture of amylase and cellulase.
